(19) United States
(12) Patent Application Publication
(10) Pub. No.: US 2020/0064358 A1
(43) Pub. Date: Feb. 27, 2020

(54) METHOD FOR PROGNOSIS OF THE OUTCOME OF PATIENTS WITH HEART FAILURE (HF)

(71) Applicants: FUNDACIÓN INSTITUTO DE INVESTIGACIÓN SANITARIA DE SANTIAGO DE COMPOSTELA, Santiago de Compostela (ES); SERVIZO GALEGO DE SAÚDE (SERGAS), Santiago de Compostela (ES); UNIVERSIDADE DE SANTIAGO DE COMPOSTELA, Santiago de Compostela (ES)

(72) Inventors: José Ramón GONZÁLEZ JUANATEY, Santiago de Compostela (ES); Rosa Mª BERMEJO AGRA, Santiago de Compostela (ES); Sonia EIRAS PENAS, Santiago de Compostela (ES)

(21) Appl. No.: 16/488,067

(22) PCT Filed: Feb. 28, 2018
(86) PCT No.: PCT/EP2018/054979

§ 371 Date: Aug. 22, 2019

(30) Foreign Application Priority Data
Feb. 28, 2017 (EP) 17382100.0

(51) Int. Cl. G01N 33/68; A61B 5/02

(52) U.S. Cl. CPC G01N 33/6893; G01N 33/6887; G01N 2800/52; G01N 2800/325; A61B 5/02

(57) ABSTRACT

The present invention offers a solution to the lack of a novel and efficient method capable to predict the outcome of patients with HF. In this context, patients with a bad prognosis are expected to be hospitalized or die in the incoming years following the diagnosis of HF, due to the HF, or due to a complication of the HF. This method is based on the concentration of Orosomucoid (APG) and Omentin in an isolated blood sample from the patient. In this sense, a high concentration of AGP and a low concentration of Omentin in blood are associated with a bad prognosis. This improved method of prognosis is aimed to help on the design of the most appropriate treatment for the patient.

### FIELD OF THE INVENTION

The present invention can be included in the field of medical prognosis of patients suffering from a specific disease, wherein specific biomarkers are used for said prognosis. More particularly, specific concentrations of proteins in the blood are used in the present invention for identifying human subjects with heart failure (HF) at risk of re-hospitalization or death in the incoming years.

### BACKGROUND OF THE INVENTION

Heart Failure (HF) is a condition in which the heart cannot pump enough blood to meet the body's needs. Patients with a new onset HF may be referred as suffering from “de novo HF”, and patients that suffer from HF from some time are often referred to as “chronic HF” patients. A treated patient with symptoms and signs that have remained generally unchanged for at least 1 month is said to be ‘stable’. If chronic stable HF deteriorates, the patient may be described as ‘decompensated’ and this may happen suddenly or slowly, often leading to hospital admission, an event of considerable prognostic importance (Ponikowski P. et al., Eur Heart J (2016) 37 (27): 2129-2200). Nowadays, HF is the leading cause of hospitalization for patients older than 65 years involving a high percentage of deaths and readmission in a short period of time (Mosterd A and Hoes A W, Heart 2007; 93(9):1137-1146).

Common treatment for heart failure includes changes in the lifestyle and medication which is generally based on:
ACE inhibitors to lower blood pressure and reduce strain on the heart. They also may reduce the risk of a future heart attack.
Aldosterone antagonists to trigger the body to remove excess sodium through urine. This lowers the volume of blood that the heart must pump.
Angiotensin receptor blockers to relax blood vessels and lower blood pressure to decrease the heart's workload.
Beta blockers to slow the heart rate and lower the blood pressure to decrease the heart's workload.
Digoxin to make the heart beat stronger and pump more blood.
Diuretics (fluid pills) to help reduce fluid buildup in your lungs and swelling in feet and ankles.
Isosorbide dinitrate/hydralazine hydrochloride to help relax blood vessels so the heart doesn't work as hard to pump blood.

However, the type and time of treatment, especially the decision about a rapid transition to advanced therapies, depends on the type and severity of the heart failure (Piotr Ponikowski et al., Eur Heart J (2016) 37 (27): 2129-2200). For instance, if there is a low risk of progression of the disease in the short term, a re-education and change in the life-style together with prescription of medicine to reduce hypertension, or in some cases prescribing ACE inhibitors or angiotensin receptor blockers, could be sufficient. However, if there are high risks of readmission due to HF, it could be advisable to add or increase diuretics, beta-blockers and/or vasodilating agents, or even consider a surgical intervention. Therefore, an accurate prognosis of the progression of the disease seems a significant measure to improve the lifespan and quality of life of patients with HF.

For diagnosis and/or prognosis, the European guidelines recommend the determination of elevated concentration values of B-type natriuretic peptide (BNP) and N-terminal pro-brain natriuretic peptide (NT-proBNP) in plasma (Ponikowski P. et al., Eur. Heart J. 2016; 37(27):2129-2200). However, their levels can be modified by other non-cardiovascular factors, such as age, renal failure (Maisel A. et al., Eur J Heart Fail 2008; 10(9):824-839) or obesity (Madamanchi C. et al., Int J Cardiol 2014; 176(3):611-617). Therefore, there is a need to identify new strategies or markers that permit to obtain a more accurate prognosis. Orosomucoid, or alpha-1-acid glycoprotein (AGP), is a protein released by the epicardial adipose tissue that has been shown to have multiple modulatory and protective properties. Recently the authors of the present invention identified AGP as a new dual indicator of mortality and/or re-hospitalization for HF in de novo and chronic HF (Agra R M et al., Int J Cardiol 2016; 228:488-494). In this sense, high AGP concentration values in plasma were identified as indicators of worse prognosis in de novo HF, and low levels for chronic HF. Omentin, also named Intelectin-1, is expressed by visceral adipose tissue and by non-fat cell from the epicardial adipose tissue. Recently, low Omentin levels were also found to be a prognosis factor in patients with HF (Narumi T. et al., Cardiovasc Diabetol 2014; 13:84). However, in the present invention, the use of both (AGP and Omentin values) is shown to be more accurate on the prognosis of patients that suffer de novo HF than using any of these parameters separately.

More specifically, high concentration of AGP together with a low concentration of Omentin in plasma is associated with a bad prognosis, i.e. with high risks of re-hospitalization for HF and/or death. Therefore, there is an improved effect by using both parameters for the prognosis of survival and/or risk of readmission in patients suffering from de novo HF.

Overall, the present invention offers a new solution to the problem of a lack of a sufficiently efficient method to make a prognosis on the progression of patients that suffer from de novo HF, preferably with similar NT-proBNP levels. This solution may also help to design a more accurate treatment for the patient.

### BRIEF DESCRIPTION OF THE INVENTION

The present invention offers a solution to the lack of a sufficiently effective method of prognosis on the outcome of patients that suffer HF. The inventors show that a low proportion (lower than 50%) of patients with HF survive without requiring re-hospitalization due to HF in the 1.5, preferably 2, even more preferably 2.5 years following the diagnosis, if they have a high concentration of AGP and a low concentration of Omentin in plasma. However, more than 90% of patients with HF survive, if they present low concentration values of AGP and high concentration values of Omentin in plasma. If they only have a high concentration of AGP or only a low concentration of Omentin in blood, more than 50% of patients survive without re-hospitalization for HF. Therefore, there is an improved effect by using both parameters (high concentration of AGP and low concentration of Omentin) for the prognosis of patients with HF. Additionally, inventors show that a high concentration of AGP together with a low concentration of Omentin in blood is directly associated with the oucome of the disease, and thus these parameters can be a cause of death or re-hospitalization for HF in the incoming years following the diagnosis. However, the levels of pro-BNP, which are commonly used for diagnosis and prognosis of patients with HF, do not appear directly related with death or re-hospitalization.

Overall, inventors propose the use of AGP and Omentin concentration levels as new effective predictors for death or re-hospitalization of patients with HF.

### BRIEF DESCRIPTION OF THE FIGURES

FIG. 1 represents a receiver operating characteristic (ROC) curve showing the ability of plasma Omentin values (ng/mL) to identify mortality or rehospitalization for HF in (A) de novo and chronic HF patients. Area 0.714; p<0.001; (B) de novo HF patients. Area 0.706; p=0.007.

FIG. 2 represents a Kaplan-Meier plots in hospitalized de novo HF patients according AGP values (> or <1.08 mg/mL) (A) or Omentin values (> or <13 ng/mL)(B). AGP values >1.08 mg/mL represented higher death or rehospitalization for HF (Log-rank p=0.008).

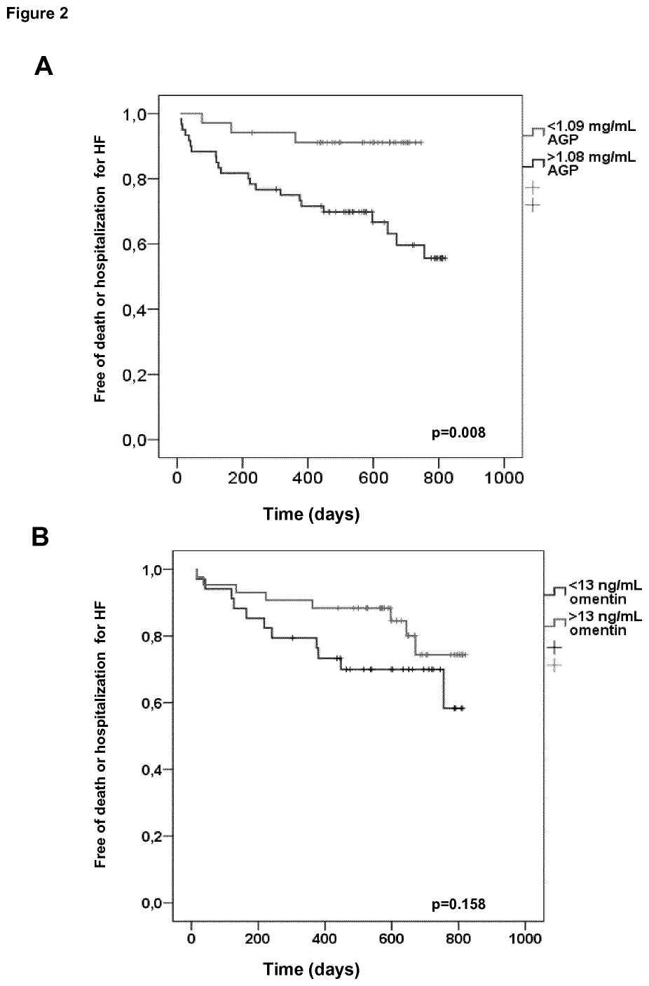

FIG. 3 represents a Kaplan-Meier plots in hospitalized de novo HF patients according AGP values (> or <1.08 mg/mL) and Omentin values (> or <13 ng/mL)(B). High AGP values >1.08 mg/mL, low AGP values <1.08 mg/mL and high Omentin values >13 ng/mL, low Omentin values <13 ng/mL (Log-rank p=0.02).

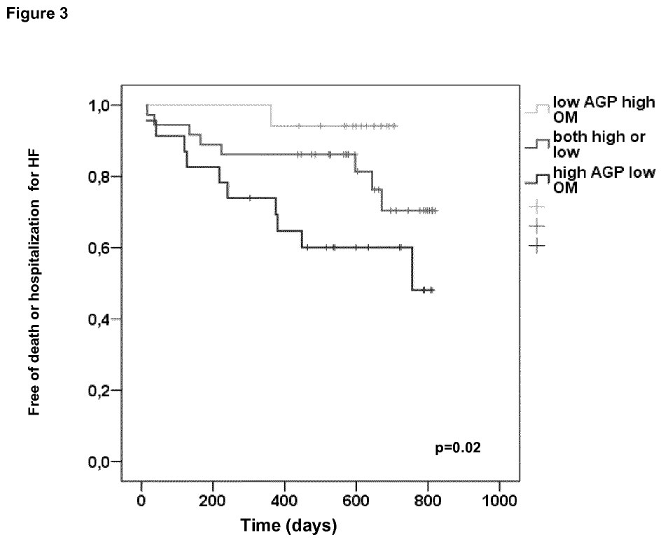

### DESCRIPTION

### Definitions

In the context of the present invention, “heart failure (HF)” refers to the well-accepted medical definition of HF. More specifically, HF is a clinical syndrome characterized by typical symptoms (e.g. breathlessness, ankle swelling and fatigue) that may be accompanied by signs (e.g. elevated jugular venous pressure, pulmonary crackles and peripheral oedema) caused by a structural and/or functional cardiac abnormality, resulting in a reduced cardiac output and/or elevated intracardiac pressures at rest or during stress. Patients with the first episode of HF are considered to be “de novo HF” patients. Patients who have had HF for some time are often considered as “chronic HF” patients.
In the context of the present invention, the term “prognosis” refers to the prospect of survival and recovery from a disease. In case of incurable disorders, prognosis may refer to the expectations of survival or of stabilization of the disease for a certain period of time. In this sense, a bad prognosis may refer to the expectation that the disease will progress so that the patient will require medical assistance or die in a certain period of time. It is anticipated from the usual course of that disease or indicated by special characteristics of the case.
In the context of the present invention, the term “diagnosis” refers to the process of determining which disease or medical condition explains a person's symptoms and signs.
In the context of the present invention, the term “biological sample” refers to a sample obtained from a biological subject, including samples of biological tissue or fluid origin obtained in vivo or in vitro. Such samples might be body fluid (i.e., blood, blood plasma, serum, or urine), but can also be organs, tissues, fractions, cells isolated from mammals including, humans and cell organelles. Biological samples also may include sections of the biological tissues from which they were obtained (i.e., sectional portions of an organ or tissue). Biological samples may also include extracts from a biological sample. Biological samples may comprise proteins, carbohydrates or nucleic acids. An isolated biological sample is a sample that is isolated from the tissue and subject from which it was obtained, and the use of said sample does not require nor imply the presence of the subject or tissue from which it was extracted, or the presence of a physician. Said isolated sample is generally processed before it is analyzed or used, so that it is different from when it was just extracted. It can also be saved for a long time under specific conditions, so that it can be analyzed and used during a long time (from hours to years) after its extraction.
In the context of the present invention, “primary antibody” refers to antibodies that specifically detect a protein of interest or target antigen, whose presence in the isolated biological sample is being analyzed. The variable region of the primary antibody is unique for this antibody, and specifically recognizes the epitope of the target antigen. The constant region of the primary antibodies can be recognized by secondary antibody that specifically recognizes this constant region.
In the context of the present invention, the term “secondary antibody” refers to antibodies that can bind to the primary antibody to assist in detection, sorting and purification of target antigens. To enable detection, the secondary antibody must have specificity for the antibody species and isotype of the primary antibody being used. The presence of this secondary antibody will then be detected by a specific reagent that will emit a signal detectable by techniques used by a skill person in the art. The presence of this signal will indirectly indicate the presence of the target antigen, or of the protein of interest, in the biological isolated sample being analyzed.
In the context of the present invention, the term “high” is used to refer to a value that is superior to another value used as reference or control. The reference levels are established prior to the analysis. A high value will be determined by a skilled person in the art. For the aspects and embodiments of the present invention, a high value refers to values that are 1.1, 1.2, 1.3, 1.4, 1.5, 1.6, 1.7, 1.8, 1.9, 2, 3, 4, 5, 6, 7, 8, 9, 10, 11, 12, 13, 14, 15, 20, 25, 30, 35, 40, 45, 50 times higher or more than the reference value.
In the context of the presence invention, the term “low” is used to refer to a value that is inferior to another value used as reference. The reference levels are established prior to the analysis. A low value will be determined by a skilled person in the art. For the aspects and embodiments of the present invention, a low value refers to values that are 1.1, 1.2, 1.3, 1.4, 1.5, 1.6, 1.7, 1.8, 1.9, 2, 3, 4, 5, 6, 7, 8, 9, 10, 11, 12, 13, 14, 15, 20, 25, 30, 35, 40, 45, 50 times lower or more than the reference value.
In the context of the present invention, the term “kit” as used herein is not limited to any specific device and includes any device suitable for working the invention such as, but not limited to, ELISA kits.
The term “comprising” it is meant including, but not limited to, whatever follows the word “comprising”. Thus, use of the term “comprising” indicates that the listed elements are required or mandatory, but that other elements are optional and may or may not be present.
By “consisting of” is meant including, and limited to, whatever follows the phrase “consisting of”. Thus, the phrase “consisting of” indicates that the listed elements are required or mandatory, and that no other elements may be present.

### DESCRIPTION OF THE INVENTION

The present invention describes the use of a new combination of parameters to establish a prognosis for patients with HF. Said combination of parameters is an inverse relation between the concentration of AGP and Omentin in the blood of the patients. In this sense, a high concentration of AGP together with a low concentration of Omentin with respect to reference values is associated with bad prognosis. In this context, patients with a bad prognosis are expected to be hospitalized or die in the 1.5, more preferably in the 2, and even more preferably in the 2.5 years, following the diagnosis of HF, due to the HF, or due to a complication of the HF, or simply because the HF progressed requiring a re-hospitalization. On the contrary, patients with a good prognosis are not expected to require a re-hospitalization or to die for any of the causes related with the HF in the 1.5, preferably in the 2, even more preferably in the 2.5 years, following the diagnosis of HF.

As indicated in the definitions, HF can be de novo or chronic HF. In all the aspects and embodiments referred herein, HF is understood to be de novo.

AGP and Omentin are proteins present in the blood. AGP is a protein released by the epicardial adipose tissue that has been shown to have multiple modulatory and protective properties. Omentin is expressed by visceral adipose tissue and by non-fat cell from the epicardial adipose tissue. The blood plasma is the liquid component of blood that normally holds the blood cells in suspension; plasma is thus the extracellular matrix of blood cells. It is mostly water (up to 95% by volume), and contains dissolved proteins (i.e. serum albumins, globulins, and fibrinogen), glucose, clotting factors, electrolytes (Na+, Ca2+, Mg2+, HCO3−, Cl−, etc.), hormones, carbon dioxide (plasma being the main medium for excretory product transportation) and oxygen. Plasma also serves as the protein reserve of the human body. It plays a vital role in an intravascular osmotic effect that keeps electrolytes in balanced form and protects the body from infection and other blood disorders. Thus, AGP and Omentin are present in the plasma of blood. Additionally, blood serum is the blood component that does not contain white or red blood cells nor clotting factors; it is the blood plasma without the fibrinogens. Serum includes all proteins from plasma not used in blood clotting (coagulation) and all of the electrolytes, antibodies, antigens, hormones, and any exogenous substances (including drugs and microorganisms or their traces). Thus, AGP and Omentin should also be present in blood serum.

A first aspect of the present invention refers to a method for predicting or prognosticating the outcome of a patient that suffers from heart failure (HF), wherein the method comprises:
a) determining the concentration of AGP and Omentin in an isolated biological sample selected from the list consisting of blood, plasma or serum, derived from the patient;
b) comparing the concentration of AGP and Omentin with reference concentration values; and
c) Assigning:
good prognosis for patients with low AGP concentration values and high Omentin concentration values in said isolated biological sample, compared with reference values, wherein good prognosis indicates that the patient is not expected to require a re-hospitalization or to die due to the HF in the 1.5, preferably in the 2, even more preferably in the 2.5 years, following the diagnosis; and
bad prognosis for patients with high AGP concentration values and low Omentin concentration values in said isolated biological sample, compared with reference values, wherein bad prognosis indicates that the patient is expected to be re-hospitalized or die due to the HF in the 1.5 years, preferably in the 2 years, even more preferably in the 2.5 years, following the diagnosis.

The method to isolate the blood plasma is known by a skilled person in the art. It generally consists on the extraction of blood cells from an isolated blood sample by centrifugation, preferably in the presence of an anticoagulant. Plasma can then be frozen until use. Serum is the liquid fraction from the whole isolated blood sample that is collected after the blood is allowed to clot. The clot is generally removed by centrifugation and the resulting supernatant, designated serum, is preferably removed using a Pasteur pipette.

Detection of AGP and Omentin in any of the isolated biological samples (blood, plasma or serum), is achieved by a method known in the art. The proteins are preferably detected in plasma, which can be diluted previously. Even more preferably, the isolated blood sample is diluted in a saline solution before the detection or even before the extraction of the plasma or the serum.

The detection of the AGP and Omentin proteins in theses isolated biological samples (blood, plasma or serum) is carried-out with a methodology selected from the list consisting of Enzyme-Linked ImmunoSorbent Assay (ELISA) method, immunohistochemistry, western blot and flow-cytometry, more preferably with ELISA. Therefore, in a first embodiment, the step a) of the method of the first aspect of the invention further comprises: detecting the proteins AGP and Omentin in the isolated blood sample and in (a) sample(s) with a known concentration of AGP and/or Omentin, using a methodology selected from the list consisting of: Enzyme-Linked ImmunoSorbent Assay (ELISA) method, immunohistochemistry, western blot and flow-cytometry. In these methods, a primary antibody specifically recognizes the protein of interest, AGP or Omentin, and a secondary antibody recognizes the constant region (Fc) of the primary antibody. The primary antibody can be monoclonal or polyclonal. The same type of secondary antibody can be used to detect both primary antibodies (anti-AGP and anti-Omentin), if the Fc region is the same for both primary antibodies. However, if the Fc region of the anti-AGP is different to that of the anti-Omentin, a specific type of secondary antibody will be used to detect each primary antibody.

The detection of the secondary antibody can be done with several reagents, preferably based on chemoluminescence, and even more preferably using the horseradish peroxidase and a substrate that, when oxidized by HRP, preferably using hydrogen peroxide as an oxidizing agent triggers a characteristic change that is detectable by spectrophotometric methods.

The determination of the concentration of AGP and Omentin in the biological sample is obtained with a method known in the art. Preferably, it may use reference samples whose concentration in Omentin and/or AGP are known. It consists on first determining the signal detected for known concentrations of said proteins in the reference samples. Then, a mathematical method, generally based on extrapolation, may be used to determine the concentration of Omentin and AGP in the isolated biological sample of interest, based on the signal detected. A computer program may be used to determine said concentration. Once determined, patients with an AGP concentration in plasma higher than 1.08 mg/ml, +−50%, preferably +−40%, more preferably +−30%, more preferably +−20%, more preferably +−15%, even more preferably +−10% and a concentration of Omentin in plasma lower than 13 ng/ml+−50%, preferably +−40%, more preferably +−30%, more preferably +−20%, more preferably +−15%, even more preferably +−10%, will have a bad prognosis. On the contrary, those patients with a concentration of AGP lower than 1.08 mg/ml+−50%, preferably +−40%, more preferably +−30%, more preferably +−20%, more preferably +−15%, even more preferably +−10% and a concentration of Omentin higher than 13 ng/ml+−50%, preferably +−40%, more preferably +−30%, more preferably +−20%, more preferably +−15%, even more preferably +−10% will have a good prognosis.

Therefore, in a preferred embodiment, the reference concentration values used to determine whether the concentration of AGP and Omentin in blood, serum or plasma are high or low in the method described in any of the previous embodiments are:
for AGP 1.08 mg/ml+−50%, preferably +−40%, more preferably +−30%, more preferably +−20%, more preferably +−15%, even more preferably +−10%.
for Omentin 13 ng/ml+−50%, preferably +−40%, more preferably +−30%, more preferably +−20%, more preferably +−15%, even more preferably +−10%.

A variety of statistical and mathematical methods for establishing the threshold, cutoff level of concentration or reference values are known in the prior art. A threshold or cutoff value of concentration for a particular biomarker may be selected, for example, based on data from Receiver Operating Characteristic (ROC) plots, as described in the Examples and Figures of the present invention. One of skill in the art will appreciate that these threshold or cutoff expression levels can be varied, for example, by moving along the ROC plot for a particular biomarker or combinations thereof, to obtain different values for sensitivity or specificity thereby affecting overall assay performance. For example, if the objective is to have a robust diagnostic method from a clinical point of view, we should try to have a high sensitivity. However, if the goal is to have a cost-effective method we should try to get a high specificity. The best cutoff refers to the value obtained from the ROC plot for a particular biomarker that produces the best sensitivity and specificity. Sensitivity and specificity values are calculated over the range of thresholds (cutoffs). Thus, the threshold or cutoff values can be selected such that the sensitivity and/or specificity are at least about 70%, and can be, for example, at least 75%, at least 80%, at least 85%, at least 90%, at least 95%, at least 96%, at least 97%, at least 98%, at least 99% or at least 100% in at least 60% of the patient population assayed, or in at least 65%, 70%, 75% or 80% of the patient population assayed.

Consequently, said predetermined reference, threshold or cutoff values correspond to the concentration value which correlates with the highest specificity at a desired sensitivity in a ROC curve calculated based on the concentration value of the protein (AGP or Omentin) determined in a patient population with HF, being at risk of dying or being re-hospitalized for HF. In this sense, concentration values of AGP higher than said reference value, and of Omentin lower than its corresponding reference value, are indicative, with said desired sensitivity, of a bad prognosis for the patient suffering from HF. In other words, they are indicative, with said desired sensitivity, of a high risk of being re-hospitalized or to die from HF in the incoming years.

The concentration of AGP and Omentin in blood plasma corresponds to another concentration in the isolated blood sample and to another one in the isolated serum fraction. When analyzing the concentration of these proteins in the isolate blood or serum samples (instead of analyzing it in the isolated blood plasma sample) the threshold, cutoff or reference values indicated above will be recalculated to adapt them to blood or serum samples. Said adaptation will be done with a method known by a person skilled in the art, such as by correlation using for example estimated values for the concentration of plasma in blood, or of serum in plasma. Said concentration of plasma in blood or of serum in plasma may also be calculated for each sample independently.

The assignment of the patient into a prognosis group can be done by a computer program, preferably, after introducing the data into said program. Thus, in another preferred embodiment, the step of assigning a good or bad prognosis according to the method described in any of the previous embodiments, is a computer implemented step wherein the data obtained in the previous steps of the method are inserted in a computer program and the program assigns the patient into one of the groups of good prognosis or bad prognosis.

This differentiation between patients may help to determine changes on the treatment. Indeed, the treatment of patients with a bad prognosis may comprise a change on the drugs administered or a change in the concentration of several drugs. Preferably, the change in the treatment might comprise adding or increasing the concentrations of diuretics, betablockers, ACEIs or ARBS, aldosterone antagonistas or advanced HF therapies that require the use of medical devices (i.e. defibrillator and/or cardiac resynchronizer), or cardiac transplant. This change on the treatment may help improve the prognosis of the patient.

A second aspect of the invention, refers to the use in vitro of reagents suitable for determining the concentration values of AGP and Omentin in an isolated biological sample selected from the list consisting of blood, plasma and serum, for prognosticating the outcome of a patient that has suffered HF, as defined in the first aspect of the invention. Said reagents might be, between others, the markers used to determine the concentration of AGP and Omentin, such as the ones described in the first aspect of the invention. Therefore, in a preferred embodiment of the second aspect of the invention, the reagents used are:
a) an antibody that specifically recognizes the protein AGP;
b) an antibody that specifically recognizes the protein Omentin;
c) a secondary antibody that specifically recognizes the antibody of a), and a secondary antibody that specifically recognizes the antibody of b), or a secondary antibody that recognizes both antibodies from a) and b), wherein the secondary antibodies can be detected with a reagent, preferably by chemoluminescence.

As indicated in the first aspect of the invention, the anti-AGP and anti-Omentin are considered primary antibodies, and can be monoclonal or polyclonal. A secondary antibody recognizes the constant region (Fc) of the primary antibody. The same type of secondary antibody can be used to detect both primary antibodies (anti-AGP and anti-Omentin), if the Fc region is the same for both. However, if the Fc region of the anti-AGP is different to that of the anti-Omentin, a specific type of secondary antibody will be used to detect each primary antibody.

The detection of the secondary antibody can be done with several reagents, preferably based on chemoluminescence, and even more preferably using the horseradish peroxidase and a substrate that, when oxidized by HRP, preferably using hydrogen peroxide as an oxidizing agent, yields a characteristic change that is detectable by spectrophotometric methods. Additional reagents may be required for the detection of the proteins, both in the isolated biological samples and also in the sample/s used as reference for determining the concentration of the proteins. Thus, in a particular embodiment of the present invention, additional reagents are used in the second aspect of the invention and are: a well-plate, a coating buffer, preferably carbonate-bicarbonate, a washing solution, preferably PBS tween 20; a blocking solution, preferably comprising TrisHCl, NaCl and BSA; a sample diluent, preferably comprising TrisHCl, NaCl, BSA and Tween 20; an enzyme substrate, preferably Tetramethylbenzidine (TMB); and a stopping solution, preferably H2SO4.

A third aspect of the present invention refers to a kit or device which comprises the reagents as defined in any of the embodiments of the second aspect of the invention.

A fourth aspect of the invention refers to the use of the kit according to the third aspect, to determine the concentration values of AGP and Omentin in an isolated biological sample selected from the list consisting of blood, serum, or plasma, for prognosticating the outcome of a patient that has suffered HF. Said prognosis will be as defined in the first aspect of the invention.

### Examples

### Material and Methods

### Study Population

This is a retrospective and observational study based on patients consecutively admitted in the Cardiology Department of the Clinical Universitary Hospital of Santiago de Compostela between May 2014 and August 2015 diagnosed with de novo HF. The exclusion criteria were decompensated chronic HF, presence of pregnancy, severe chronic liver or renal disease, autoimmune or chronic inflammatory diseases, recent (last 3 weeks) infectious process, recent (last 3 weeks) treatment with corticosteroids or antiinflammatory drugs, known tumor processes at the time of inclusion in the study or blood disorders.

The database collected demographic, clinical (electrocardiogram and echocardiogram parameters within 24 hours after admission), laboratory analysis (haemogram, basic biochemistry and coagulation rate, lipid and glucose profile). Specific parameters were also registered after admission, such as the levels of glycosylated haemoglobin, albumin, electrolytes and pro brain natriuretic peptide (proBNP). The study complies with the Declaration of Helsinki and was approved by the Clinical Research Ethics Committee of Galicia. All patients provided an informed consent. Heart failure diagnosis was made according to the recommendations of the European Society of Cardiology (Ponikowski P et al., Eur Heart J 2016; 37(27):2129-2200).

### Omentin-1 and Orosomucoid Analysis

Blood samples from 76 patients admitted for HF were obtained at discharge. Blood samples from patients were centrifuged at 1800×g for 15 minutes. Isolated plasma was stored at −80° C. until use. Plasma was diluted 100 times and Omentin levels were measured with ELISA kit with a detection limit of 6.5 pg/mL (SEA933Hu, Cloud Clone Corp, Houston, USA) following manufacture's protocol. Orosomucoid levels were analysed as previously described (Agra R M et al., Int J Cardiol 2016; 228:488-494).

### Endpoint Definition and Follow-Up

The endpoints were death from any cause and re-hospitalization for HF. Follow-up information was recorded from medical history. The mean of follow-up was 521 (9-820) days.

### Statistical Analysis

Clinical characteristics of all patients were expressed as mean±standard deviation (SD) for continues variables or as percentage (%) for the categorical variables. Differences among the determined groups of patients were expressed similarly. The comparison between groups was analyzed with a one-way ANOVA test for variables with a normal distribution and with Kruskal-Wallis test in those skewed. Comparison of categorical variables among subgroups was performed by a chi-squared test. Cut-off values for Omentin levels were obtained from the receiver operating characteristic (ROC) curve. Log-rank test was used to compare the probability of survival or rehospitalization for HF among groups with the Omentin and AGP levels in de novo HF. Univariate and multivariate Cox regression analyses were used to calculate the estimated hazard ratio (HR) with 95% confidence interval (CI), where appropriate. The variables were entered into a multivariate model for factors with a p value ≤0.05 in the univariate analysis. The Statistical Package for Social Science (SPSS) for Windows, version 15.0 (software SPSS Inc.; Chicago, Ill., USA) package, was used for all statistical analyses. Statistical significance was defined as p<0.05.

### Results

### Baseline Characteristics of Patients

A total of 76 patients (mean age 68 years, 57% men) were admitted in our cardiology department for de novo HF. 54 of them were diganosed with hypertension (71%); 35 with type 2 diabetes mellitus (T2DM) (46%); 15 with ischemic aetiology (19.7%). The mean left ventricular ejection fraction (LVEF) was 42%, the hemoglobin levels were 13.6±1.6 and the proBNP was 4183±4783 as Table 1.

TABLE 1
Clinical characteristics of the included patients

| Sex (men), n (%) | 44 | (57) |
|---|---|---|
| Age, years (mean ± SD) | 68.8 ± 12.08 | |
| BMI (kg/m2), (mean ± SD) | 30.3 ± 6.7 | |
| HTA, n (%) | 54 | (71) |
| HLP, n (%) | 39 | (51) |
| T2DM, n (%) | 35 | (46.1) |
| Ischaemic Etiology, n (%) | 15 | (19.7) |
| Systolic blood pressure(mmHg), mean ± SD | 140.2 ± 29.9 | |
| Diastolic blood pressure (mmHg), mean ± SD | 86.3 ± 19.8 | |
| Heart rate at admission, bpm, mean ± SD | 102.7 ± 28.5 | |
| LVEF %, mean ± SD | 41.2 ± 14.6 | |
| ACEI, n (%) | 51 | (67.1) |
| Betablockers, n (%) | 61 | (80.3) |
| MRAs, n (%) | 64 | (84) |
| Statins, n (%) | 43 | (56.6) |
| Diuretics, n (%) | 73 | (96.1) |

| | |
|---|---|
| Hb (g/dl), mean ± SD | 13.6 ± 1.6 |
| Glucose (mg/dl), mean ± SD | 144.7 ± 59.4 |
| Creatinine (mg/dl), mean ± SD | 1.02 ± 0.39 |
| Sodium (meq/l) | 141.3 ± 4.2 |
| Potasium (meq/l) | 4.5 ± 0.6 |
| proBNP (pg/mL), mean ± SD | 4183 ± 4783 |
| AGP-1 (mg/mL), mean ± SD | 1.65 ± 0.9 |
| Omentin (mg/mL), mean ± SD | 13.6 ± 5.3 |

BMI: Body mass index;
LVEF: Left ventricle ejection fraction;
T2DM: Type 2 diabetes mellitus;
HTA: Hypertension;
HLP: Hyperlipemia;
proBNP: pro-Brain natriuretic peptide;
ACEI-ARBs: Angiotensin converting enzyme inhibitor-Angiotensin receptor blockers;
MRA: Mineralocorticoids receptor antagonists;
AGP: Alpha 1-acid glycoprotein;
Hb; Hemoglobin;
SD: Standar deviation

### Omentin and AGP Cut-Off Values Determination for Death and Rehospitalization for HF.

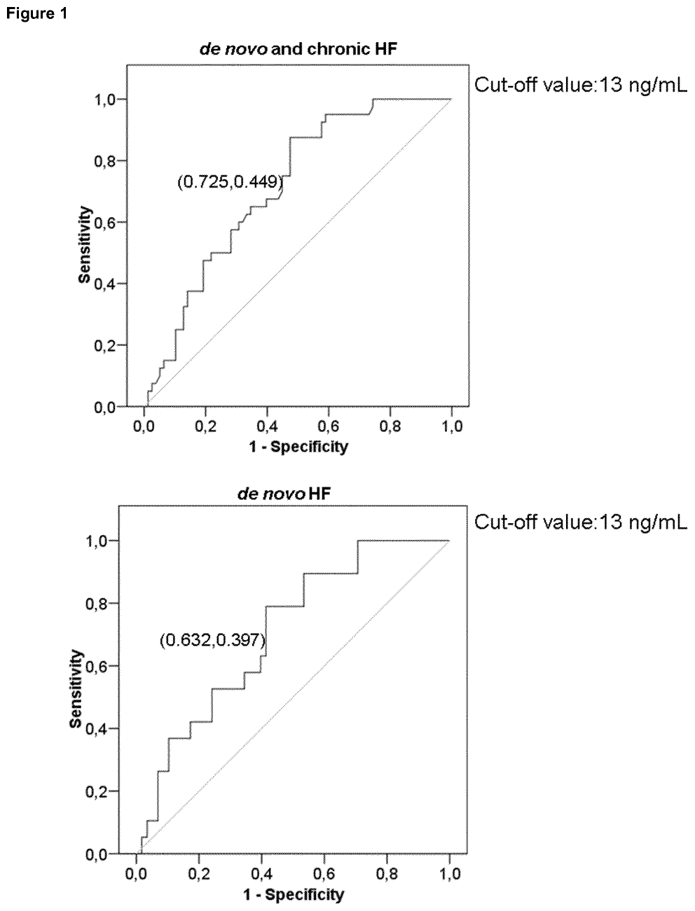

The cut off values of AGP and Omentin for determining death or readmission for HF were calculated with an area under the ROC curve (AUC). The AGP cut off value was 1.08 mg/ml in blood palsma, as previously described by our group (Agra R M et al., Int J Cardiol 2016; 228:488-494). Omentin cut off values for HF patients (acute de novo and chronic) performed an area under the curve (AUC) of 0.714 with a 95% confidence interval (CI), 0.622-0.806; p<0.001; and 13 ng/ml on blood plasma of this protein was associated with a sensitivity of 0.72 and a specificity of 0.55. After selecting only patients with de novo HF, Omentin values performed an area under the curve (AUC) with a lower statistical significance: 0.706 with 95% confidence interval (CI), 0.581-0.830; p=0.007 and 13 ng/mL of Omentin was associated with sensitivity of 0.62 and a specificity of 0.60 (FIG. 1). Thus, a high AGP concentration was considered for concentration values higher than 1.08 mg/mL, and a high Omentin concentration was considered for concentration values higher than 13 ng/mL.

### Differences Among Groups of Patients According Omentin and AGP Values

Patients were stratified according AGP-1 and Omentin levels. Thus, three groups were established. One group was for patients with high AGP-1 and low Omentin concentration levels, another group for patients with low AGP-1 and high Omentin concentration levels, and another group was formed by patients with AGP-1 and Omentin concentration levels both high or both low. The comparison among groups indicated that those patients with high AGP-1 and low Omentin levels had lower ejection fraction (Table 2). However, there were no statistically significant differences regarding proBNP values.

TABLE 2
Differences among groups of patients according Omentin and AGP values
(statistical analysis used described in materials and methods).

| | AGP low, | | AGP and Omentin | | AGP high, | | |
|---|---|---|---|---|---|---|---|
| | Omentin high | | (both high or both low) | | Omentin low | | |
| | n = 17 | | n = 36 | | n = 23 | | p |
| Sex (men), n (%) | 8 | (47.1) | 21 | (58.3) | 15 | (65.2) | 0.515 |
| Age, years (mean ± | 68.5 ± 13.8 | | 68.6 ± 10.9 | | 69.5 ± 12.8 | | 0.950 |
| SD) | | | | | | | |
| BMI (kg/m2), (mean ± | 29.3 ± 4.2 | | 31.7 ± 6.8 | | 28.9 ± 7.8 | | 0.240 |
| SD) | | | | | | | |
| HTA, n (%) | 15 | (88.2) | 25 | (69.4) | 14 | (60.9) | 0.162 |
| HLP, n (%) | 10 | (58.8) | 17 | (47.2) | 12 | (52.2) | 0.729 |
| T2DM, n (%) | 9 | (52.9) | 11 | (30.6) | 5 | (21.7) | 0.106 |
| Ischaemic Etiology, | 2 | (11.8) | 7 | (19.4) | 6 | (26.1) | 0.530 |
| (%) | | | | | | | |
| Systolic blood | 138.9 ± 25.8 | | 143.1 ± 31.5 | | 136.6 ± 31.0 | | 0.709 |
| pressure(mmHg), | | | | | | | |
| mean ± SD | | | | | | | |
| Diastolic blood | 89.8 ± 17.6 | | 86.6 ± 21.4 | | 83.2 ± 19.1 | | 0.579 |
| pressure (mmHg), | | | | | | | |
| mean ± SD | | | | | | | |
| Heart rate at | 102.9 ± 27.9 | | 102.3 ± 26.8 | | 103.1 ± 32.6 | | 0.995 |
| admission, bpm, | | | | | | | |
| mean ± SD | | | | | | | |
| LVEF %, mean ± SD | 42.1 ± 15.9 | | 45.2 ± 14.0 | | 34.2 ± 12.5 | | 0.016 |
| ACEI, n (%) | 14 | (82.4) | 18 | (50) | 19 | (82.6) | 0.011 |
| Betablockers, n (%) | 14 | (82.4) | 29 | (80.6) | 18 | (78.3) | 0.948 |
| MRAs, n (%) | 13 | (76.5) | 29 | (80.6) | 22 | (95.7) | 0.183 |
| Statins, n (%) | 9 | (52.9) | 21 | (58.3) | 13 | (56.5) | 0.934 |
| Diuretics, n (%) | 17 | (100) | 34 | (94.4) | 22 | (95.7) | 0.621 |

| | | | | |
|---|---|---|---|---|
| Hb (g/dl), mean ± SD | 13.2 ± 1.5 | 13.7 ± 1.6 | 13.8 ± 1.4 | 0.395 |
| Glucose (mg/dl), | 166.0 ± 71.7 | 129.2 ± 49.8 | 152.7 ± 59.3 | 0.083 |
| mean ± SD | | | | |
| Creatinine (mg/dl), | 0.79 ± 0.1 | 1.08 ± 0.45 | 1.08 ± 0.34 | 0.025 |
| mean ± SD | | | | |
| Sodium (meq/l) | 141.5 ± 2.2 | 141.3 ± 4.7 | 141.0 ± 4.5 | 0.990 |
| Potasium (meq/l) | 4.3 ± 0.6 | 4.5 ± 0.6 | 4.5 ± 0.5 | 0.359 |
| proBNP (pg/mL), | 4650 ± 5340 | 3325 ± 2938 | 5186 ± 6431 | 0.328 |
| mean ± SD | | | | |
| AGP-1 (mg/mL), | 0.74 ± 0.23 | 1.87 ± 0.92 | 1.99 ± 0.74 | <0.001 |
| mean ± SD | | | | |
| Omentin (mg/mL). | 17.32 ± 2.91 | 14.67 ± 5.63 | 9.45 ± 3.18 | <0.001 |
| mean ± SD | | | | |

BMI: Body mass index;
LVEF: Left ventricle ejection fraction;
T2DM: Type 2 diabetes mellitus;
HTA: Hypertension;
HLP: Hyperlipemia;
proBNP: pro-Brain natriuretic peptide;
ACEI-ARBs: Angiotensin converting enzyme inhibitor-Angiotensin receptor blockers;
MRA: Mineralocorticoids receptor antagonists;
AGP: Alpha 1-acid glycoprotein;
Hb; Hemoglobin;
SD: Standar deviation

### Uni and Multivariate Analyses to Predict Death or HF Readmission

In the univariate analysis, we studied all the parameters related with the combined outcome (death or readmission for HF). Among these parameters, those that appeared to better predict mortality or readmission for HF were age HR, Cl 95% 1.06 (1.01-1.12), heart rate HR, Cl 95% 0.97 (0.96-1.00), high proBNP HR, Cl 95% 1.00 (1.00-1.01), creatinine HR, Cl 95% 3.64 (1.40-9.45) and high AGP and low Omentin levels HR, Cl 95% 2.55 (1.24-5.24) in patients with AHF, as shown in table 3a. However, in a multivariate analysis only AGP-Omentin remained as an independent predictor value of death or rehospitalization for HF (Table 3b). Thus, the levels of APG and Omentin together appear to be the best predictors for death or rehospitalization after de novo HF.

TABLE 3
Uni and multivariate analyses to predict death or HF readmission

| | |
|---|---|
| HR, CI 95% | p |

A) Univariate analysis

| | | |
|---|---|---|
| Age | 1.06 (1.01-1.12) | 0.017* |
| Sex | 1.42 (0.57-3.53) | 0.446 |
| Heart rate (at admision) | 0.97 (0.96-1.00) | 0.006* |
| Systolic blood pressure | 1.00 (0.99-1.02) | 0.629 |
| LVEF | 1.00 (0.97-1.04) | 0.760 |
| Hb | 0.85 (0.64-1.13) | 0.255 |
| Sodium | 0.99 (0.89-1.10) | 0.811 |
| Creatinine | 3.64(1.40-9.45) | 0.008* |
| proBNP | 1.00 (1.00-1.00) | 0.010* |
| High AGP-Low Omentin | 2.55 (1.24-5.24) | 0.011* |

B) Multivariate analysis (COX regression)

| | | |
|---|---|---|
| Age | 1.03 (0.97-1.08) | 0.345 |
| Heart rate | 0.98 (0.96-1.00) | 0.076 |
| Creatinine | 2.77 (0.84-9.08) | 0.093 |
| proBNP | 1.00 (1.00-1.00) | 0.692 |
| High AGP-Low Omentin | 2.20 (1.02-4.75) | 0.045* |

LVEF: Left ventricle ejection fraction;
AGP: Alpha 1-acid glycoprotein;
Hb; Hemoglobin;
HR: Hazard Ratio;
CI: Confidence Interval;
*p ≤ 0.05

1. A method for prognosticating the outcome of a patient that suffers from de novo heart failure (HF), wherein the method comprises:
a. determining the concentration of Orosomucoid (AGP) and Omentin in an isolated biological sample selected from the list consisting of blood, plasma and serum, obtained from the patient;
b. comparing the concentration of AGP and Omentin with reference concentration values; and
c. assigning:
i. good prognosis for patients with low AGP concentration values and high Omentin concentration values in said isolated biological sample, compared with reference concentration values, wherein good prognosis indicates that the patient is not expected to require a re-hospitalization or to die due to the HF in the 2.5 years following the diagnosis; and
ii. bad prognosis for patients with high AGP concentration values and low Omentin concentration values in said isolated biological sample, compared with reference concentration values, wherein bad prognosis indicates that the patient is expected to be re-hospitalized or to die due to the HF in the 2.5 years following the diagnosis.

2. The method according to claim 1, wherein step a) further comprises: determining the concentration of proteins AGP and Omentin in the isolated sample and in sample(s) with a known concentration of AGP and/or Omentin, using a methodology selected from the list consisting of: Enzyme-Linked ImmunoSorbent Assay (ELISA) method, immunohistochemistry, western blot and flow-cytometry.

3. The method according to any of the preceding claims, wherein the reference concentration values used to determine whether the concentration of AGP and Omentin in blood plasma are high or low are:
a. for AGP the reference concentration value is 1.08 mg/ml; and
b. for Omentin the reference concentration value is 13 ng/ml.

4. The method according to any of the preceding claims, wherein step c) is a computer implemented step wherein the data obtained in the previous steps are inserted in a computer program and the program assigns the patient into one of the groups of good prognosis or bad prognosis.

5. Use in vitro of reagents suitable for determining the concentration values of AGP and Omentin in an isolated biological sample selected from the list consisting of blood, serum and plasma, for prognosticating the outcome of a patient that suffers de novo HF, wherein good or bad prognosis is as defined in claim 1.

6. The use according to claim 5, wherein the reagents are:
a. an antibody that specifically recognizes the protein AGP;
b. an antibody that specifically recognizes the protein Omentin;
c. a secondary antibody that specifically recognizes the antibody of a), and a secondary antibody that specifically recognizes the antibody of b), or a secondary antibody that recognizes both antibodies from a) and b), wherein the secondary antibodies can be detected with a reagent, preferably by chemoluminescence.

7. The use according to any of claims 5 to 6, wherein further reagents are: a well-plate, a coating buffer, preferably carbonate-bicarbonate, a washing solution, preferably PBS tween 20; a blocking solution, preferably comprising TrisHCl, NaCl and BSA; a sample diluent, preferably comprising TrisHCl, NaCl, BSA and Tween 20; an enzyme substrate, preferably Tetramethylbenzidine (TMB); and a stopping solution, preferably H2SO4.

8. A kit which comprises reagents suitable for determining the concentration values of AGP and Omentin.

9. The kit according to claim 8, wherein said kit comprises the reagents as defined in any of claims 6 or 7.

10. Use in vitro of the kit according to any of claims 8 or 9, to determine the concentration values of AGP and Omentin in an isolated biological sample selected from the list consisting of blood, plasma and serum, for prognosticating the outcome of a patient that suffers de novo HF wherein good or bad prognosis is as defined in claim 1.
